SEQUENCE LISTING (4 sequences)

SEQ ID NO: 1 - DNA, 27 bp, Artificial Sequence
  Primer-1
atcacgcatatggcgaaaatactttgc

SEQ ID NO: 2 - DNA, 30 bp, Artificial Sequence
  Primer-2
atagaattcttatcagccggccttcttgaa

SEQ ID NO: 3 - DNA, 43 bp, Artificial Sequence
  Primer-3
actgaattctaaggaggttaacaatggaactttttaaatatat

SEQ ID NO: 4 - DNA, 32 bp, Artificial Sequence
  Primer-4
gatgagctcttattaacgtctgcttaatacac

(19) United States
(12) Patent Application Publication
(10) Pub. No.: US 2022/0025351 A1
(43) Pub. Date: Jan. 27, 2022

(54) PRODUCTION METHOD FOR IMMOBILIZED MICROORGANISMS AND PRODUCTION METHOD FOR AMINO ACID USING THE SAME

(71) Applicant: KANEKA CORPORATION, Osaka-shi, Osaka (JP)

(72) Inventors: Mirai TANAKA, Takasago-shi (JP); Hiroyuki KANAMARU, Takasago-shi (JP); Noriyuki ITO, Takasago-shi (JP); Hiroaki YASUKOUCHI, Takasago-shi (JP)

(73) Assignee: KANEKA CORPORATION, Osaka-shi, Osaka (JP)

(21) Appl. No.: 17/276,222

(22) PCT Filed: Sep. 3, 2019
(86) PCT No.: PCT/JP2019/034560

§ 371 Date: Mar. 15, 2021

(30) Foreign Application Priority Data
Sep. 18, 2018 (JP) 2018-174075

(51) Int. Cl. C12N 11/12; C12P 13/04

(52) U.S. Cl. CPC C12N 11/12; C12P 13/04; C12Y 101/99033

(57) ABSTRACT

An object of the present invention is to provide a method for producing an immobilized microorganism having high filtration properties, and to provide a method for producing an amino acid using the immobilized microorganism. A method for producing an immobilized microorganism is characterized in that a microorganism is contacted with carboxymethyl cellulose sodium salt and then contacted with polyethylenimine and an alkane dial after the first contact with carboxymethyl cellulose sodium salt.

### TECHNICAL FIELD

The present invention provides a method to immobilize a microorganism.

### BACKGROUND ART

Though material conversion reaction using a microorganism and an enzyme can realize the reaction with high selectivity more effectively under ordinary temperature and ordinary pressure, it is not easy to separate target material from the used microorganism and enzyme in the reaction. It is therefore suggested that a microorganism and an enzyme to be used can be immobilized on a polymer to form an immobilized enzyme or an immobilized microorganism. By using immobilized enzyme or immobilized bacterium, it becomes easier to separate a target substance formed by the reaction by an operation such as filtration. In addition, a serial reaction such as a production of a target substance advantageously becomes possible by filling a column with an immobilized enzyme or an immobilized bacterium and flowing a liquid containing a raw material through the column.

For example, non-patent document 1 discloses that an immobilized cell is obtained by dispersing an E. coli cell collected by centrifuging of a culture broth into water, adding polyethylenimine thereto, collecting a resultant aggregate by centrifugation, re-dispersing the aggregate in a potassium phosphate buffer solution, adding glutaraldehyde thereto , stirring the mixture, and performing freeze-dry and pulverization.

### RELATED ART DOCUMENTS

### Non-Patent Document

Non-patent document 1: ITOH et al., Continuous production of chiral 1,3-butanediol using immobilized biocatalysts in a packed bed reactor: promising biocatalysis method with an asymmetric hydrogen-transfer bioreduction, Applied Microbiol and Biotechnol, Springer Science+Business Media, Germany, vol. 75, 2007(pp. 1249-1256)

### DISCLOSURE OF THE INVENTION

### Problems to be Solved by the Invention

It was however found that the filtration property of an immobilized cell is low in the method of Non-patent document 1. A productivity on an industrial scale becomes low when an immobilized microorganism has a low filtration property.

The present invention is completed under the above-described circumstances. The objective of the present invention is to provide a method to produce an immobilized microorganism having higher filtration property and to provide a method to produce an amino acid using the immobilized microorganism.

### Means for Solving the Problems

The present invention which can solve the problem above-described is as follows.

[1] A method for producing an immobilized microorganism, comprising the steps of:

contacting a microorganism with carboxymethyl cellulose sodium salt, and then

further contacting the microorganism with polyethylenimine and an alkane dial.

[2] The production method according to [1], wherein the microorganism is first contacted with the polyethylenimine and then contacted with the alkane dial after the microorganism is contacted with the carboxymethyl cellulose sodium salt.

[3] The production method according to [1] or [2], wherein each contact was made in the presence of a dispersion medium comprising water.

[4] The production method according to any one of [1] to [3], wherein a viscosity of the carboxymethyl cellulose sodium salt measured by the following condition is 50 mPa·s or less.

Viscosity Measurement Condition:

a 2% aqueous solution is prepared by precisely weighing 4.4 g of carboxymethyl cellulose sodium salt, adding the weighed carboxymethyl cellulose sodium salt into a 300 mL stoppered conical flask, determining an amount (W) of water by the following formula, and adding the determined amount (W) of water:

Required water amount W(g)=carboxymethyl cellulose sodium salt(g)×(98−water content (%))/2

wherein the water content (%) is a water content in the carboxymethyl cellulose sodium salt and corresponds to a weight loss on drying in the case where the carboxymethyl cellulose sodium salt is dried in a constant temperature dryer of 105±2° C. for 4 hours;

the prepared 2% carboxymethyl cellulose sodium salt aqueous solution is left to stand overnight and then stirred using a magnetic stirrer for 5 minutes to obtain a complete solution, the complete solution is added into a covered container having a diameter of 45 mm and a height of 145 mm, the container is immersed in a constant temperature bath of 25±0.2° C. for 30 minutes, the complete solution is slowly stirred using a glass bar after a temperature of the complete solution becomes 25° C., a rotor and a guard of a BM-type viscometer are installed, a scale is read off 3 minutes after the rotor is rotated at a rotation speed of 30 rpm or 60 rpm; and

the viscosity value (mPa·s) is calculated by multiplying the following coefficient determined with the Rotor No. and the rotation speed by the read scale.

Coefficient in the case of Rotor No. 1 and 60 rpm: 1

Coefficient in the case of Rotor No. 2 and 60 rpm: 5

Coefficient in the case of Rotor No. 3 and 60 rpm: 20

Coefficient in the case of Rotor No. 4 and 60 rpm: 100

Coefficient in the case of Rotor No. 1 and 30 rpm: 2

Coefficient in the case of Rotor No. 2 and 30 rpm: 10

Coefficient in the case of Rotor No. 3 and 30 rpm: 40

Coefficient in the case of Rotor No. 4 and 30 rpm: 200

[5] The production method according to any one of [1] to [4], wherein the microorganism is recombinant Escherichia coli.

[6] The production method according to [5], wherein the recombinant Escherichia coli is a transformant having an amino acid dehydrogenase activity.

[7] The production method according to [5], wherein the recombinant Escherichia coli is a transformant having a leucine dehydrogenase activity and a formate dehydrogenase activity.

[8] A method for producing an amino acid, comprising the steps of:

producing the immobilized microorganism by the method according to any one of [1] to [7], and

contacting the immobilized microorganism with a keto acid.

[9] The method for producing the amino acid according to [8], wherein a column is filled with the immobilized microorganism, a solution comprising the keto acid is supplied to an inlet of the column, and a solution comprising the amino acid is discharged from an outlet of the column.

[10] The method for producing the amino acid according to [8] or [9], wherein the keto acid is 3,3-dimethyl-2-oxobutyric acid and the amino acid is tert-leucine.

In this description, “immobilization” means the condition where a microorganism and polyethylenimine are forming a complex and the microorganism doesn't get away from the complex even when the complex is washed with an eluent, especially water.

### Effects of the Invention

An immobilized microorganism having a high filtration property can be produced by the present invention.

### Mode for Carrying Out the Invention

The present invention method is characterized in that the first step to contact the microorganism to be immobilized with carboxymethylcellulose sodium salt and the second step to contact the obtained mixture with polyethylenimine and an alkane dial are conducted. A filtration property of the obtained immobilized microorganism is improved in accordance with the method for producing an immobilized microorganism, the method comprising the first step and the second step, an aggregate in an appropriate condition such as an appropriate size, hardness and figure is formed from the microorganism and carboxymethylcellulose sodium salt in the first step and the immobilization may be progressed by polyethylenimine and the alkane dial on the basis of the generated aggregate in the second step. Hereinafter, the aggregate is referred to as a microorganism-CMC complex in some cases.

First Step

As the microorganism used to be immobilized, a prokaryote such as Escherichia coli and an eukaryote such as yeast are available, Escherichia coli is preferred, and recombinant Escherichia coli is more preferred. Recombinant Escherichia coli is applicable to produce an immobilized microorganism having desired activity efficiently.

The carboxymethylcellulose sodium salt used in the first step is preferably has a viscosity of 50 mPa·s or less, more preferably 30 mPa·s or less, and even more preferably 20 mPa·s or less. The lower limit of the viscosity is not particularly restricted and may be, for example 1 mPa·s or more. The most preferable viscosity value of the carboxymethylcellulose sodium salt is 5 mPa·s or more and 12 mPa·s or less.

The viscosity above means the value decided by the following viscosity measurement method.

Viscosity Measurement Condition:

a 2% aqueous solution is prepared by precisely weighing 4.4 g of carboxymethyl cellulose sodium salt, adding the weighed carboxymethyl cellulose sodium salt into a 300 mL stoppered conical flask, determining an amount (W) of water by the following formula, and adding the determined amount (W) of water:

Required water amount W(g)=carboxymethyl cellulose sodium salt(g)×(98−water content (%))/2

wherein the water content (%) is a water content in the carboxymethyl cellulose sodium salt and corresponds to a weight loss on drying in the case where the carboxymethyl cellulose sodium salt is dried in a constant temperature dryer of 105±2° C. for 4 hours;

the prepared 2% carboxymethyl cellulose sodium salt aqueous solution is left to stand overnight and then stirred using a magnetic stirrer for 5 minutes to obtain a complete solution, the complete solution is added into a covered container having a diameter of 45 mm and a height of 145 mm, the container is immersed in a constant temperature bath of 25±0.2° C. for 30 minutes, the complete solution is slowly stirred using a glass bar after a temperature of the complete solution becomes 25° C., a rotor and a guard of a BM-type viscometer are installed, a scale is read off 3 minutes after the rotor is rotated at a rotation speed of 30 rpm or 60 rpm; and

the viscosity value (mPa·s) is calculated by multiplying the following coefficient determined with the Rotor No. and the rotation speed by the read scale.

Coefficient in the case of Rotor No. 1 and 60 rpm: 1

Coefficient in the case of Rotor No. 2 and 60 rpm: 5

Coefficient in the case of Rotor No. 3 and 60 rpm: 20

Coefficient in the case of Rotor No. 4 and 60 rpm: 100

Coefficient in the case of Rotor No. 1 and 30 rpm: 2

Coefficient in the case of Rotor No. 2 and 30 rpm: 10

Coefficient in the case of Rotor No. 3 and 30 rpm: 40

Coefficient in the case of Rotor No. 4 and 30 rpm: 200

An etherification degree of the carboxymethylcellulose sodium salt may be, for example, 0.3 or more, preferably 0.6 or more, more preferably 0.7 or more, and for example, may be 3.0 or less, preferably 1.5 or less, more preferably 1.0 or less, the most preferably 0.8 or less.

An amount of the carboxymethylcellulose sodium salt to 100 parts by mass of the dried microorganism may be, for example, 1 part by mass or more, preferably 10 parts by mass or more, more preferably 20 parts by mass or more, and for example, may be 5000 part by mass or less, preferably 1000 parts by mass or less, more preferably 500 parts by mass or less.

The contact of microorganism with the carboxymethylcellulose sodium salt is preferably carried out in the presence of a dispersant. Such a dispersant makes the aggregation condition of the microorganism-CMC complex appropriate for filtration.

The dispersant preferably contains at least water and is exemplified by water and a mixed solvent of water and the other solvent. Hereinafter, such a dispersant is referred to as an aqueous dispersant. One kind of the other solvent to be mixed with water may be used, or a plurality of the other solvents may be used. The use of the aqueous dispersant enables the microorganism-CMC complex to aggregate in better condition.

The other solvent is preferably a water-soluble solvent and more preferably a solvent having a compatibility with water in all composition range. An example of the water-soluble solvent includes an ether solvent such as tetrahydrofuran, 1,4-dioxane and t-butyl methyl ether; a ketone solvent such as acetone, methyl ethyl ketone and cyclohexanone; an alcohol solvent such as methanol, ethanol, isopropanol and benzylalcohol. The other solvent is preferably tetrahydrofuran, 1,4-dioxane, acetone and an aliphatic alcohol, and more preferably methanol, ethanol and isopropanol.

An amount of water in the dispersant to total 100 parts by mass of water and the other solvent may be, for example, 30 parts by mass or more, preferably 60 parts by mass or more, and more preferably 90 parts by mass or more.

A total amount of the microorganism and the carboxymethylcellulose sodium salt in the first step to 100 parts by mass of the dispersant may be, for example, 0.1 parts by mass or more, preferably 0.5 parts by mass or more, more preferably 1 part by mass or more, and for example, 100 parts by mass or less, preferably 10 parts by mass or less, more preferably 5 parts by mass or less.

A procedure to contact the microorganism and the carboxymethylcellulose sodium salt is not particularly restricted, and a liquid prepared by dispersing or dissolving, preferably dissolving, the carboxymethylcellulose sodium salt in an appropriate dispersant is preferably added, preferably dropwise, to a liquid prepared by dispersing the microorganism in order to improve an aggregation condition. The above dispersant is preferably water or a mixed solvent of water and the other solvent, and particularly preferably water. The above liquid prepared by dispersing or dissolving the carboxymethylcellulose sodium salt in the appropriate dispersant is, for example, a liquid prepared dissolving the carboxymethylcellulose sodium salt in water, and is referred to as a CMC liquid in some cases. The above liquid prepared by dispersing the microorganism is referred to as a microorganism suspension in some cases.

A concentration of the microorganism in 100 mass % of the microorganism suspension may be, for example, 0.1 mass % or more, preferably 0.5 mass % or more, more preferably 1 mass % or more, and for example, may be less than 100 mass %, preferably 10 mass % or less, more preferably 5 mass % or less.

A concentration of the carboxymethylcellulose sodium salt in 100 mass % of the CMC liquid may be, for example, 0.5 mass % or more, preferably 1 mass % or more, more preferably 2 mass % or more, and for example, may be 15 mass % or less, preferably 10 mass % or less, more preferably 7 mass % or less.

An adding time, preferably a dropwise adding time, of the CMC liquid may be, for example, 10 minutes or more, preferably 20 minutes or more, and for example, may be 2 hours or less, preferably 1 hour or less, more preferably 40 minutes or less.

A liquid containing both components prepared by contacting the microorganism and the carboxymethylcellulose sodium salt may be stirred to be mixed for an appropriate time. The microorganism and the carboxymethylcellulose sodium salt are preferably mixed, and the CMC liquid is more preferably added dropwise to be mixed. The stirring time may be, for example, minutes or more, preferably 20 minutes or more, more preferably 30 minutes or more, and for example, may be 5 hours or less, preferably 3 hours or less, more preferably 1 hour or less.

When the liquid containing both of the microorganism and the carboxymethylcellulose sodium salt is stirred to be mixed, the power required for stirring may be, for example, 0.001 kW/m³ or more, preferably 0.01 kW/m³ or more, more preferably 0.1 kW/m³ or more, and for example, may be 100 kW/m³ or less, preferably 50 kW/m³ or less, more preferably 10 kW/m³ or less.

Both of the temperature of the microorganism suspension when the CMC liquid is added dropwise and the temperature when the liquid containing both of the microorganism and the carboxymethylcellulose sodium salt is stirred to be mixed may be respectively, for example, 5° C. or higher, preferably 10° C. or higher, more preferably 15° C. or higher, and for example, may be 50° C. or lower, preferably 40° C. or lower, more preferably 30° C. or lower.

Second Step

After obtaining the mixture of the microorganism and the carboxymethylcellulose sodium salt, i.e. microorganism-CMC complex, in the first step, the microorganism-CMC complex is contacted with polyethylenimine and an alkane dial in the second step. It is preferred that the microorganism-CMC complex is first contacted with polyethylenimine, and then contacted with alkane dial.

The above polyethylenimine may be a linear polyethylenimine of which all amino groups are secondary amino groups or a branched polyethylenimine having both of a secondary amino group and a tertiary amino group, and is preferably the branched polyethylenimine. The branched polyethylenimine preferably further has a primary amino group.

A molecular weight of the polyethylenimine may be, for example, 1000 or more, preferably 10000 or more, more preferably 50000 or more, and for example, may be 1000000 or less, preferably 500000 or less, more preferably 100000 or less. The above molecular weight may be measured or may be alternatively a molecular weight described in a catalogue thereof.

The polyethylenimine is preferably a liquid, and the viscosity thereof measured at 25° C. may be, for example, 200 mPa·s or more, preferably 400 mPa·s or more, more preferably 600 mPa·s or more, and for example, may be 1100 mPa·s or less, preferably 1000 mPa·s or less, more preferably 900 mPa·s or less.

An amount of the polyethylenimine to 100 parts by mass of the dried microorganism may be, for example, 0.1 parts by mass or more, preferably 0.5 parts by mass or more, more preferably 10 parts by mass or more, even more preferably 50 parts by mass or more, and for example, may be 1000 parts by mass or less, preferably 500 parts by mass or less, more preferably 100 parts by mass or less, even more preferably 20 parts by mass or less, even more preferably 10 parts by mass or less, particularly preferably 5 parts by mass.

An example of the alkane dial includes an alkane dial having the carbon number of about 3 or more and about 10 or less, such as malondialdehyde, 1,4-butanedial, glutaraldehyde as 1,5-pentanedial, 1,6-hexanedial, 2,5-dimethylhexanedial. The alkane dial is preferably a linear C4-6 alkane dial and more preferably glutaraldehyde.

An amount of the alkane dial to 100 parts by mass of the dried microorganism may be, for example, 1 part by mass or more, preferably 5 parts by mass or more, more preferably 10 parts by mass or more, and for example, may be 10000 parts by mass or less, preferably 1000 parts by mass or less, more preferably 200 parts by mass or less.

The microorganism-CMC complex is preferably contacted with polyethylenimine and alkane dial in the presence of a dispersant. By the presence of such a dispersant, the microorganism-CMC complex can be appropriately immobilized, and the filtration property can be further improved. The dispersant used in the second step can be selected from the similar range of the dispersant used in the first step and is preferably the same as the dispersant used in the first step.

A procedure to contact the microorganism-CMC complex with polyethylenimine and alkane dial is not particularly restricted, a liquid prepared by dispersing or dissolving, preferably dissolving, polyethylenimine in a dispersant and a liquid prepared by dispersing or dissolving, preferably dissolving, the alkane dial are added, preferably dropwise, to the microorganism-CMC complex in terms of a further improvement of the filtration property after immobilization. The dispersant for polyethylenimine is preferably water or a mixed solvent of water and the other solvent, and particularly preferably water. The liquid of polyethylenimine is, for example, a liquid prepared by polyethylenimine in water, and is hereinafter referred to as a polyethylenimine liquid in some cases. The dispersant for the alkane dial is preferably water or a mixed solvent of water and the other solvent, and particularly preferably water. The liquid of the alkane dial is, for example, a liquid prepared by the alkane dial in water, and is hereinafter referred to as an alkane dial liquid in some cases. A liquid containing both of polyethylenimine and alkane dial may be added, preferably dropwise, and it is preferred that polyethylenimine and alkane dial is respectively added, preferably dropwise.

A concentration of polyethylenimine in the polyethylenimine liquid may be, for example, 1 mass % or more, preferably 5 mass % or more, more preferably 10 mass % or more, and for example, may be 50 mass % or less, preferably 40 mass % or less, more preferably 30 mass % or less.

The polyethylenimine liquid is preferably neutralized. A pH value of the polyethylenimine liquid may be, for example, 9.0 or less, preferably 8.0 or less, more preferably 7.5 or less, and for example, may be 5.0 or more, preferably 6.0 or more, more preferably 6.5 or more.

A time to add, preferably add dropwise, the polyethylenimine liquid may be, for example, 10 minutes or more, preferably 20 minutes or more, and for example, may be 2 hours or less, preferably 1 hour or less, more preferably 40 minutes or less.

An alkane dial concentration in the alkane dial liquid may be, for example, 10 mass % or more, preferably 20 mass % or more, more preferably 30 mass % or more, and for example, may be 80 mass % or less, preferably 70 mass % or less, more preferably 60 mass % or less.

A time to add, preferably add dropwise, the alkane dial liquid may be, for example, 10 minutes or more, preferably 20 minutes or more, and for example, may be 2 hours or less, preferably 1 hour or less, more preferably 40 minutes or less.

When the polyethylenimine liquid and the alkane dial liquid are separately added, it is preferred that the polyethylenimine liquid is first added and then the alkane dial liquid is added. It is preferred that after the addition of the polyethylenimine liquid, the mixture is stirred for a certain time as so called aging and then the addition of the alkane dial liquid starts.

A time for the above-described aging may be, for example, 10 minutes or more, preferably 20 minutes or more, and for example, may be 2 hours or less, preferably 1 hour or less, more preferably 40 minutes or less.

After the addition of both of the polyethylenimine liquid and the alkane dial liquid is completed, the mixture is preferably subjected to aging. The aging time after the addition of both of the polyethylenimine liquid and the alkane dial liquid may be, for example, 10 minutes or more, preferably 20 minutes or more, and for example, may be 2 hours or less, preferably 1 hour or less, more preferably 40 minutes or less.

A temperature to stir each liquid in the second step may be, for example, 5° C. or higher, preferably 10° C. or higher, more preferably 15° C. or higher, and for example, may be 50° C. or lower, preferably 40° C. or lower, more preferably 30° C. or lower.

A liquid in which the microorganism is immobilized, hereinafter referred to as an immobilized microorganism liquid in some cases, is obtained by mixing all of the microorganism, carboxymethylcellulose sodium salt, polyethylenimine solution and alkane dial. A concentration of the immobilized microorganism in the immobilized microorganism liquid is described as a mass of the dried immobilized microorganism to 100 parts by mass of the total immobilized microorganism liquid. The mass of the dried immobilized microorganism may be, for example, 0.001 parts by mass or more, preferably 0.01 parts by mass or more, 0.1 parts by mass or more, and for example, may be 99 parts by mass or less, preferably 95 parts by mass or less, more preferably 90 parts by mass or less.

Isolation Step

The immobilized microorganism liquid is washed as needed and filtered to isolate the immobilized microorganism. For example, water and a buffer solution having a pH of 5 or more and 8 or less can be used for the washing, and the buffer solution is preferably tris(hydroxymethyl)aminomethane, i.e. Tris. The washing procedure is not particularly restricted, and a common method may be adopted. For example, a pulping by a washing liquid and a separation by precipitation such as centrifugation or filtration may be repeated.

Immobilized Microbial Property

The thus obtained immobilized microorganism has excellent filtration property and excellent productivity on an industrial scale. In addition, the activity of the microorganism is kept high. The immobilized microorganism is excellent in liquid permeability and volume-maintaining property; and even when a column is filled with the immobilized microorganism to be used for a reaction, a performance loss is small.

Filtration property can be evaluated by specific filtration resistance in the case where a Tris buffer solution containing the immobilized microorganism is filtrated using a filter paper 5A manufactured by Kiriyama Glass Works Co. having an area of 15.2 cm² in a condition of a filtration pressure of 1.0 kgf/m³ and a cake thickness of 3 cm. When the immobilized microorganism of the present invention is used, the specific filtration resistance becomes, for example, 5×10¹¹ m/kg or less, preferably 5×10¹⁰ m/kg or less, and more preferably 5×10⁹ m/kg or less.

An activity-maintaining property of the microorganism is described as an activity yield, specifically an activity of the microorganism after the immobilization on the assumption that an activity of the microorganism before the immobilization is 1. The activity-maintaining property may be, for example, 0.1 or more, preferably 0.2 or more, and more preferably 0.3 or more. The activity property is preferably 1 and may be 0.8 or less or 0.6 or less.

A liquid permeability is evaluated by a pressure loss measured by filling a pressure proof glass column (Omnifit) having an internal diameter of 10 mm with about 9 g of the immobilized microorganism, putting the column in a column oven controlled at 30° C., standing and fixing the column upright, and then supplying distilled water into the column. A pressure loss in the case where the immobilized microorganism of the present invention is used and distilled water is flowed at 500 cm/hr may be 0.5 MPa or less, preferably 0.1 MPa or less, and the most preferably 0.05 MPa or less.

A volume-maintaining property is an index affected by a falling prevention ability of the microorganism from the immobilized microorganism and a consolidation durability, and is evaluated by a volume change measured by filling a pressure proof glass column (Omnifit) having an internal diameter of 10 mm with about 9 g of the immobilized microorganism, putting the column in a column oven controlled at 30° C., standing and fixing the column upright, and then supplying distilled water into the column at the space velocity, SV of 0.45 hr⁻¹ for 56 hours. The volume change measured by using the immobilized microorganism of the present invention may be, for example, 0.8 or more, preferably 0.9 or more, and more preferably 0.95 or more. The volume change corresponds to a volume measured by flowing a liquid for 56 hours on the assumption that a volume measured by flowing a liquid for 1 hour is 1 as a standard.

Reaction by Immobilized Microorganism

The immobilized microorganism can be used for various reactions depending on the microorganisms to be immobilized. For example, when the microorganism, particularly recombinant Escherichia coli, has an amino acid dehydrogenases activity, an amino acid can be obtained by contacting the immobilized microorganism with a keto acid.

The above-described amino acid dehydrogenase is an enzyme having the activity to reductively aminate a keto acid or a cyclic imine. An example of the amino acid dehydrogenase includes phenylalanine dehydrogenase, leucine dehydrogenase, and pyrroline-2-carboxylate reductase, and a leucine dehydrogenase is preferable.

An amino acid dehydrogenase can be obtained from a microorganism having the amino acid dehydrogenase production ability. An example of the microorganism having the amino acid dehydrogenases production ability includes a microorganism belonging to a genus of Brevibacterium, Rhodococcus, Sporosarcina, Thermoactinomyces, Microbacterium, Halomonas, Clostridium, Bacillus, Neurospora, Escherichia and Aerobacter, a microorganism belonging to a genus of Bacillus is preferable, and Bacillus badius IAM11059 and Bacillus sphaericus NBRC3341 are more preferable.

When the above microorganism, particularly recombinant Escherichia coli, has an amino acid dehydrogenase activity, the microorganism preferably has a coenzyme-regenerative activity. A reaction by an amino acid dehydrogenase needs a reduced coenzyme such as NADH, and NADH is converted to an oxidized form, i.e. NAD⁺, as a reaction proceeds. When the microorganism has the coenzyme-regenerative activity to convert an oxidized coenzyme to a reduced coenzyme, an amount of a coenzyme usage can be reduced.

An example of the enzyme having a coenzyme-regenerative activity includes hydrogenase, formate dehydrogenase, alcohol dehydrogenase, aldehyde dehydrogenase, glucose 6-phosphate dehydrogenase and glucose dehydrogenase, and a formate dehydrogenase is preferable.

A formate dehydrogenase can be obtained from a microorganism having the formate dehydrogenase production ability. An example of such a microorganism having the formate dehydrogenase production ability includes a microorganism belonging to a genus of Candida, Kloeckera, Pichia, Lipomyces, Pseudomonas, Moraxella, Hyphomicrobium, Paracoccus, Thiobacillus, Ancylobacter. Preferably a microorganism belonging to a genus of Thiobacillus and Ancylobacter, and more preferably Thiobacillus sp. KNK65MA (FERM BP-7671) and Ancylobacter aquaticus KNK607 (FERN BP-7335).

As a recombinant Escherichia coli having both an amino acid dehydrogenase activity, especially a leucine dehydrogenase activity, and a formate dehydrogenase activity includes Escheria coli HB101 (pFT 001) (FERN BP-7672) and Escheria coli HB101 (pFT 002) (FERN BP-7673). Each Escheria coli above have the amino acid dehydrogenase gene derived from Bacillus sphaericus NBRC3341 and the formate acid dehydrogenase gene derived from Thiobacillus sp. KNK65MA (FERN BP-7671). WO 05/090950 pamphlet can be referred to as to Escheria coli HB101 (pFT 001) (FERN BP-7672) and Escherichia coli HB101 (pFT002) (FERM BP-7673).

When a keto acid is treated with the microorganism having an amino acid dehydrogenase activity, the keto acid is reductively aminated and an amino acid is obtained. The keto acid is preferably an α-keto acid, more preferably a chemical compound shown in formula (1).

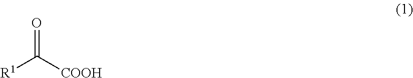

In the formula, R¹ represents a C₁₋₂₀ alkyl group which may have a substituent, a C₇₋₂₀ aralkyl group which may have a substituent, or a C₆₋₂₀ aryl group which may have a substituent.

A C₁₋₂₀ alkyl group includes, for example, methyl group, isopropyl group, isobutyl group, 1-methylpropyl group, carbamoylmethyl group, 2-carbamoylmethyl group, hydroxyethyl group, 1-hydroxyethyl group, mercaptomethyl group, 2-methylthioethyl group, (1-mercapto-1-methyl)ethyl group, 4-aminobutyl group, 3-guanidinopropyl group, 4(5)-imidazolemethyl group, ethyl group, n-propyl group, n-butyl group, t-butyl group, 2,2-dimethylpropyl group, chloromethyl group, methoxymethyl group, 2-hydroxyethyl group, 3-aminopropyl group, 2-cyanoethyl group, 3-cyanopropyl group, 4-(benzoylamino)butyl group and 2-methoxycarbonylethyl group.

A C₇₋₂₀ aralkyl group is not particularly restricted, benzyl group, indolylmethyl group, 4-hydroxybenzyl group, 2-fluorobenzyl group, 3-fluorobenzyl group, 4-fluorobenzyl group and 3,4-methylenedioxybenzyl group are included.

A C₆₋₂₀ aryl group includes phenyl group and 4-hydroxyphenyl group.

A substituent includes amino group, hydroxyl group, nitro group, cyano group, carboxyl group, alkyl group, aralkyl group, aryl group, alkanoyl group, alkenyl group, alkynyl group, alkoxyl group and halogen.

A keto acid is the most preferably 3,3-dimethyl-2-oxobutanoic acid which is called trimethylpyruvic acid.

By treating with the microorganism having an amino acid dehydrogenase activity, an α-amino acid is obtained from an α-keto acid, preferably an α-L-amino acid is obtained. An α-L-amino acid in formula (2) is obtained from an α-keto acid in formula (1) and a tert-leucine is obtained from 3,3-dimethiy-2-oxobutanoic acid.

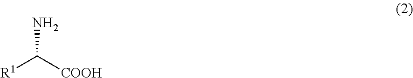

In the formula, R¹ means the same as above.

The immobilized microorganism obtained by immobilizing a microorganism having an amino acid dehydrogenase activity by above-mentioned method, hereinafter referred to as an immobilized microorganism to be treated with a keto acid, is reacted with a keto acid by contact in the presence of a solvent.

The reaction solvent is selected from the similar range of the dispersant used in the first step to immobilize a microorganism, preferably water or a mixed solvent of water and other solvent, more preferably water. A concentration of a keto acid may be adjusted to, for example, 1 mass % or more, preferable 5 mass % or more, more preferably 10 mass % or more, and for example, 90 mass % or less, preferably 60 mass % or less, more preferably 40 mass % or less.

A reaction temperature may be, for example, 10° C. or higher, preferably 20° C. or higher, more preferably 25° C. or higher, and for example, may be 80° C. or lower, preferably 60° C. or lower, more preferably 40° C. or lower.

A reaction is preferably carried out within a specific range of a pH adjusted by using a buffer material or adding acid or base, then the pH may be, for example, 4 or more, preferable 6 or more, more, and for example, may be 12 or less, preferably 10 or less, more preferably 8 or less.

When a keto acid and the immobilized microorganism to be treated with a keto acid are reacted, a coenzyme like NADH preferably coexists. Also, when the immobilized microorganism to be treated with a keto acid has a coenzyme-regenerative activity, a coenzyme like NAD⁺ preferably coexists. With the presence of a coenzyme, a reaction efficiency is improved. A dosage of a coenzyme to a keto acid may be, for example, 0.000001 equivalent or more, preferably 0.00001 equivalent or more, more preferably 0.0001 equivalent or more, and for example, may be 2 equivalent or less, preferably 0.1 equivalent or less, more preferably 0.01 equivalent or less.

When a keto acid and the immobilized microorganism to be treated with a keto acid are reacted, a chemical compound which contributes to regenerate a coenzyme preferably coexists, the chemical compound may be, for example, hydrogen, formic acid, alcohol, aldehyde compounds and glucose. A dosage of a chemical compound which can contribute to regenerate a coenzyme to a keto acid may be, for example, 0.1 equivalent or more, preferably 0.5 equivalent or more, more preferably 1 equivalent or more, and for example, may be 100 equivalent or less, preferably 50 equivalent or less, more preferably 10 equivalent or less.

The reaction above is conducted by a batch method, preferably a continuous flow method, more preferably a continuous flow method with column reactor where a liquid containing a raw material is supplied from the inlet of a column and a liquid containing the reaction product is discharged from the outlet of a column. A microorganism is immobilized having high activity, a high liquid permeability and a high volume-maintaining property by the method of the present invention, therefore the immobilized microorganism in column reactor is suitable for an immobilized catalyst by using continuous flow method.

A fluid flow velocity of a liquid containing a raw material in a column type continuous flow reaction may be at the space velocity, SV, for example, 0.1 hr⁻¹ or more, preferably 0.2 hr⁻¹ or more, more preferably 0.3 hr⁻¹ or more, and for example, may be 3 hr⁻¹ or less, preferably 2 hr⁻¹ or less, more preferably 1 hr⁻¹ or less.

A reaction product obtained by using an immobilized microorganism is isolated and refined as needed. A common separation method can be used preferably in combination, the method may be, for example, extraction, thickening, crystallization and column chromatography.

The present application claims the benefit of the priority dates of Japanese patent application No. 2018-174075 filed on Sep. 18, 2018. All of the contents of the Japanese patent application No. 2018-174075 filed on Sep. 18, 2018, are incorporated by reference herein.

### EXAMPLES

Hereinafter, the examples are described to demonstrate the present invention more specifically, but the present invention is in no way restricted by the examples, and the examples can be appropriately modified to be carried out within a range which adapts to the contents of this specification. Such a modified example is also included in the range of the present invention.

### Production Example 1: Production of Culture Medium for Bacterium

Production of the plasmid designed to have the ability to express a volume of formate dehydrogenase:

PCR reaction was performed using the genomic DNA of Thiobacillus sp. KNK65MA (FERM BP-7671) as a template and primers under the PCR condition 1 shown below. Primer 1 is SEQ ID NO: 1 and Primer 2 is SEQ ID NO: 2 in the sequence listing.

PCR Condition 1

For the preparation of a reaction solution, 1.25 U, equivalent to 0.25 μL, of Pyrobest DNA polymerase manufactured TAKARA BIO INC., 5 μL of 10×Pyrobest Buffer II manufactured by TAKARA BIO INC., 4 μL from each 2.5 mM dNTP solution, 2 μL from each 20 μM primer solution were added to 100 ng of the DNA template. Distilled water was added to the mixture so that the total volume became 50 μL. The PCR reaction was consisted of three steps, a denaturation step conducted at 96° C. for 30 seconds, an annealing step conducted at 50° C. for 30 seconds, an extension step conducted at 72° C. for 90 seconds, and the PCR sample was cooled down to 4° C. after the cycle was repeated for 25 times.

The DNA fragment amplified via PCR was digested with restriction endonuclease NdeI and EcoRI and the digested fragment was ligated to the vector plasmid pUCNT with T4 DNAligase. A person with an ordinary skill in the art can produce pUCNT on the basis of WO 94/03613 pamphlet. A plasmid designed to have the ability to express a volume of formate dehydrogenase was obtained.

Production of the Plasmid Designed to have the Ability to Express a Volume of Leucine Dehydrogenase:

PCR reaction was performed using the genomic DNA of Bacillus sphaericus NBRC3341 as a template and primers under the above-described PCR condition 1. Primer 3 is SEQ ID NO: 3 and Primer 4 is SEQ ID NO: 4 in the sequence listing.

The fragment amplified via PCR was digested with restriction endonuclease EcoRI and Sad and the digested fragment was ligated to vector plasmid pUCNT with T4 DNAligase. A person with an ordinary skill in the art can produce pUCNT on the basis of WO 94/03613 pamphlet. The plasmid pUCNT had disrupted recognition site of NdeI by receiving one base replacement. A plasmid designed to have the ability to express a volume of leucine dehydrogenase was obtained.

The obtained plasmid designed to have the ability to express a volume of leucine dehydrogenase was digested with restriction endonuclease EcoRI and PstI, and the DNA fragment having leucine dehydrogenase gene was recovered by using TaKaRa RECOCHIP manufactured by TAKARA BIO INC.

The above plasmid designed to have the ability to express a volume of formate dehydrogenase was digested at the recognition sites of EcoRI and PssI located in the downstream of formate dehydrogenase gene, and then DNA fragment was obtained. The DNA fragment was ligated with the above-described DNA fragment having leucine dehydrogenase gene by T4 DNA ligase to obtain a plasmid designed to have the ability to express a volume of both the leucine dehydrogenase and the formate dehydrogenase.

The competent cell of Escherichia coli HB101 was transformed by mixing with the obtained plasmid, and the transformant having both the leucine dehydrogenase activity and the formate dehydrogenase activity was bred.

The bred transformant having both the leucine dehydrogenase activity and the formate dehydrogenase activity was inoculated to sterilized culture medium A which contained tryptone 1.6%, yeast extract 1.0%, sodium chloride 0.5% and Ampicillin 0.01% and which prepared by dissolving the components other than Ampicillin in deionized water to obtain a solution having pH of 7.0, sterilizing the solution, and adding Ampicillin thereto, and then the mixture was shaken at 33° C. for 48 hours to aerobically cultivate the transformant.

### Example 1: Production of Immobilized Bacterium

In order to remove 1166 g of supernatant, 1740 g of culture medium of which wet bacteria mass was 35 g and which was obtained in Production example 1 was centrifuged. To the stirred 580 g of residual concentrated culture medium at room temperature, 145 g of 5 mass % aqueous solution of carboxymethylcellulose sodium named Serogen6A and manufactured by DKS. Co. Ltd., was added over 20 minutes. The solution was stirred for another 30 minutes. Then, 66 g of 20% polyethylenimine aqueous solution of which pH was adjusted to 7 using hydrochloric acid was added to the stirred solution at room temperature over 20 minutes. The polyethylenimine aqueous solution called “EPOMIN” p-1000 is manufactured by Nippon Shokubai Co., Ltd. and has 70000 of molecular weight described in a catalog. The mixed solution was stirred for another 30 minutes. To the stirred solution at room temperature, 24 g of 50 mass % glurataldehyde aqueous solution was added for over 20 minutes. The solution was stirred for another 30 minutes. Stirring was stopped, and the solution was allowed to stand for about 5 minutes to generate precipitation. After the supernatant was removed using a pipet, 290 mL of 50 mM Tris-HCl adjusted to pH 7.5 was added thereto, and the mixture was stirred at room temperature for 30 minutes. The above procedure was repeated two more times.

The obtained mixed liquid was filtrated with a filter paper 5 A manufactured by Kiriyama Glass Works Co. having an area of 15.2 cm² in a condition of a filtration pressure of 1.0 kgf/m² and a cake thickness of 3 cm, the filtration property was excellent as the filtration rate resistance coefficient was 1.5×10⁹ m/kg. In this way, 63 g of immobilized bacterium was obtained.

A pressure proof glass column (Omnifit) having an internal diameter of 10 mm was filled with about 9 g of the obtained immobilized bacterium, put into a column oven controlled at 30° C., standing and fixing the column upright, and then distilled water was supplied into the column using a syringe. It could be confirmed that a column with a low pressure loss can be produced, since distilled water could run through the column without applying a high pressure on the syringe.

### Example 2: Evaluation of Immobilized Vacterium's Activity Yield

To 1 mL of a reaction mixture of which pH was adjusted to 7.3 as a 50 mM potassium phosphate buffer solution containing 200 mg of trimethyl pyruvate, 1.45 mg of NAD⁺, 20 mg of zinc sulfate heptahydrate, 150 mg of ammonium formate and 60 mg of ammonium sulfate, 200 mg of the obtained immobilized bacterium or 9.2 mL of the obtained concentrated culture medium ultrasonically pulverized was added. The mixture was stirred at 30° C. for 1 hour. The yield and optical purity of the remained trimethyl pyrate and produced L-tert-Leucine were analyzed with high-performance liquid chromatography, HPLC, as a result, the activity yield obtained by dividing total activity of immobilized bacterium by total activity of culture medium used for immobilization of the immobilized bacterium was 0.4.

HPCL analysis condition for trimethyl pyruvate

Column: COSMOSIL 5C18-AR, 4.6 mm×250 mm in size, manufactured by NACALAI TESQUE, INC.

Mobile phase: 10 mM potassium phosphate buffer solution adjusted to pH 2.0/acetonitrile=95/5 (V/V)

Flow rate: 1 mL/min

Column temperature: 40° C.

Detection: 210 nm

HPCL analysis condition for L-tert-Leucine

Column: SUMICHIRAL OA-5000, 4.6 mm×250 mm in size, manufactured by Sumika Chemical Analysis Service, Ltd.

Mobile phase: 2 mM copper sulfate aqueous solution/methanol=95/5 (V/V)

Flow rate: 1 mL/min

Column temperature: 35° C.

Detection: 254 nm

### Comparative Example 1: Production of Immobilized Bacterium

In order to remove 1166 g of supernatant, 1740 g of culture medium of which wet bacteria mass was 35 g and which was obtained in Production example 1 was centrifuged. To the stirred 580 g of residual concentrated culture medium at room temperature, 66 g of 20 mass % polyethylenimine called “EPOMIN” and manufactured by Nippon Shokubai Co., Ltd. aqueous solution of which pH was adjusted to 7 by a hydrochloric acid was added over 20 minutes. The solution was stirred for another 30 minutes. To the stirred solution at room temperature, 24 g of 50 mass % glurataldehyde aqueous solution was added for over 20 minutes. The solution was stirred for another 30 minutes. Stirring was stopped, and the solution was allowed to stand for about 5 minutes to generate precipitation. After the supernatant was removed using a pipet, 290 ml of 50 mM Tris-HCl adjusted to pH 7.5 was added thereto, and the mixture was stirred at room temperature for 30 minutes. The above procedure was repeated two more times.

The obtained mixed liquid was filtered with a filter paper 5A manufactured by Kiriyama Glass Works Co. having an area of 15.2 cm² under reduced pressure of 15 mmHg; as a result it was confirmed that efficient production of immobilized bacterium was difficult.

Instead of filtrating the mixed liquid, immobilized bacterium was obtained by using a large amount of paper towel to absorb liquid component of the mixed liquid. A pressure proof glass column (Omnifit) having an internal diameter of 10 mm was filled with about 9 g of the obtained immobilized bacterium, put in a column oven controlled at 30° C., standing and fixing the column upright, and then, distilled water was supplied into the column using a syringe. It was confirmed that the pressure loss became huge when a column was packed with the immobilized microorganism, since higher pressure was needed to allow distilled water to pass through the column.

### Comparative Example 2: Synthesis of L-tert-Leucine by Batch Method

In a glass reaction vessel, 102.1 mg of immobilized bacterium obtained in Comparative example 1 was mixed with each 10 ml of Solution A and Solution B. After the mixture was stirred for 16 hours at room temperature, a sample of the reaction mixture was analyzed with HPLC; as a result, mole conversion ratio from trimethyl pyruvate to L-tert-Leucine was 20.3%.

Preparation Method of Solution A

To a trimethyl pyruvate solution of 5.80 g (66 wt %), 6 N NaOH aqueous solution and 50 mM potassium phosphate buffer solution were added, and pH of the solution was adjusted to 7. Then, the volume of the solution was increased to 20 mL with 50 mM potassium phosphate.

Preparation Method of Solution B

After NAD⁺ of 2.9 mg, zinc sulfate heptahydrate of 4.0 mg, ammonium formate of 3.0 g, ammonium sulfate of 1.2 g and 1 ml of 1 M potassium phosphate buffer solution adjusted to pH 7 were mixed, the volume of the solution was increased to 20 mL with distilled water.

### Example 3: Synthesis of L-tert-Leucine by Flow Method 1

A pressure proof glass column (Omnifit) having an internal diameter of 10 mm was filled with 2.98 g of the immobilized bacterium obtained in Example 1, put in a column oven controlled at 30° C., standing and fixing the column upright, and then distilled water was pumped into the column at a flow rate of 0.05 ml/min with a syringe pump manufactured by YMC.CO., LTD. Subsequently, a raw material solution prepared by mixing 19 ml each of Solution B and Solution C was pumped into the column at a flow rate of 0.03 ml/min, SV of 0.45 hr⁻¹, with a syringe pump manufactured by YMC.CO., LTD. for 68 hours in total to obtain a reaction mixture containing the L-tert-leucine from the column outlet. HPLC recovery rate was 99%. Mole conversion ratios of the obtained reaction mixture at times of 23, 46 and 68 hours were 97%, 99% and 97% respectively.

Preparation Method of Solution C

Distilled water of 2.9 g was added to trimethyl pyruvate aqueous solution of 2.90 g (66 wt %). Then, the pH of the solution was adjusted to 7 with 6 N NaOH aqueous solution and 50 mM potassium phosphate buffer solution, and finally the volume of the solution was increased to 20 mL with 50 mM potassium phosphate buffer solution.

### Example 4: Synthesis of L-tert-Leucine by Flow Method 2

A pressure proof glass column (Omnifit) having an internal diameter of 10 mm was filled with 8.94 g of the immobilized bacterium obtained in Example 1, put in a column oven controlled at 30° C., standing and fixing the column upright, and then distilled water was pumped into the column at a flow rate of 0.1 ml/min with a plunger pump manufactured by FLOM corporation. A raw material solution prepared by mixing 140 ml each of Solution D and Solution E was pumped into the column with a plunger pump manufactured by FLOM Corp. at a flow rate of 0.09 ml/min, SV of 0.45 hr⁻¹, for 56 hours totally, and the reaction mixture remaining in the column was extruded by pumping distilled water into the column at the same flow rate. As a result, the reaction mixture containing 11.9 g of the L-tert-leucine was obtained, and mole conversion rate was 99% and HPLC recovery rate was 87%. Mole conversion rates of the obtained reaction mixture at times of 20, 26, and 44 hours were all 99%. Moreover, the height of the immobilized bacterium filled in the column didn't change from 5.1 cm measured at the start of the reaction, it was considered that the volume of the immobilized bacterium didn't change before and after the reaction and no elution of the bacterium from the immobilized bacterium occurred and the carrier for the immobilization was not dissolved.

Preparation Method of Solution D

To trimethyl pyruvate solution of 20.3 g (70 wt %), distilled water of 20.3 g was added. The solution was adjusted to pH 7 with 6 N NaOH aqueous solution and 50 mM potassium phosphate buffer solution. To the solution, 50 mM potassium phosphate buffer solution was added so that the total volume became 140 mL.

Preparation Method of Solution E

After NAD⁺ of 20.3 mg, zinc sulfate heptahydrate of 28.0 mg, ammonium formate of 21.0 g, ammonium sulfate of 8.4 g and 7 ml of 1 M potassium phosphate buffer solution adjusted to pH 7 were mixed, distilled water was added thereto so that the total volume became 140 mL.

### INDUSTRIAL APPLICABILITY

An immobilized catalyst having an excellent property can be produced by the present invention in a simple way, and the present invention can be advantageously utilized for various synthesis reaction, preferably an enantioselective synthesis reaction.

1. A method for producing an immobilized microorganism, comprising the steps of:
contacting a microorganism with carboxymethyl cellulose sodium salt, and then further contacting the microorganism with polyethylenimine and an alkane dial.

2. The production method according to claim 1, wherein the microorganism is first contacted with the polyethylenimine and then contacted with the alkane dial after the microorganism is contacted with the carboxymethyl cellulose sodium salt.

3. The production method according to claim 1, wherein each contact was made in the presence of a dispersion medium comprising water.

4. The production method according to claim 1, wherein a viscosity of the carboxymethyl cellulose sodium salt measured by the following condition is 50 mPa·s or less,
Viscosity measurement condition:
a 2% aqueous solution is prepared by precisely weighing 4.4 g of carboxymethyl cellulose sodium salt, adding the weighed carboxymethyl cellulose sodium salt into a 300 mL stoppered conical flask, determining an amount (W) of water by the following formula, and adding the determined amount (W) of water:
Required water amount W(g)=carboxymethyl cellulose sodium salt(g)×(98−water content(%))/2
wherein the water content (%) is a water content in the carboxymethyl cellulose sodium salt and corresponds to a weight loss on drying in the case where the carboxymethyl cellulose sodium salt is dried in a constant temperature dryer of 105±2° C. for 4 hours;
the prepared 2% carboxymethyl cellulose sodium salt aqueous solution is left to stand overnight and then stirred using a magnetic stirrer for 5 minutes to obtain a complete solution, the complete solution is added into a covered container having a diameter of 45 mm and a height of 145 mm, the container is immersed in a constant temperature bath of 25±0.2° C. for 30 minutes, the complete solution is slowly stirred using a glass bar after a temperature of the complete solution becomes 25° C., a rotor and a guard of a BM-type viscometer are installed, a scale is read off 3 minutes after the rotor is rotated at a rotation speed of 30 rpm or 60 rpm; and
the viscosity value (mPa·s) is calculated by multiplying the following coefficient determined with the Rotor No. and the rotation speed by the read scale,
Coefficient in the case of Rotor No. 1 and 60 rpm: 1
Coefficient in the case of Rotor No. 2 and 60 rpm: 5
Coefficient in the case of Rotor No. 3 and 60 rpm: 20
Coefficient in the case of Rotor No. 4 and 60 rpm: 100
Coefficient in the case of Rotor No. 1 and 30 rpm: 2
Coefficient in the case of Rotor No. 2 and 30 rpm: 10
Coefficient in the case of Rotor No. 3 and 30 rpm: 40
Coefficient in the case of Rotor No. 4 and 30 rpm: 200.

5. The production method according to claim 1, wherein the microorganism is recombinant Escherichia coli.

6. The production method according to claim 5, wherein the recombinant Escherichia coli is a transformant having an amino acid dehydrogenase activity.

7. The production method according to claim 5, wherein the recombinant Escherichia coli is a transformant having a leucine dehydrogenase activity and a formate dehydrogenase activity.

8. A method for producing an amino acid, comprising the steps of:
producing the immobilized microorganism by the method according to claim 1, and
contacting the immobilized microorganism with a keto acid.

9. The method for producing the amino acid according to claim 8, wherein a column is filled with the immobilized microorganism, a solution comprising the keto acid is supplied to an inlet of the column, and a solution comprising the amino acid is discharged from an outlet of the column.

10. The method for producing the amino acid according to claim 8, wherein the keto acid is 3,3-dimethyl-2-oxobutyric acid and the amino acid is tert-leucine.
